(19) United States
(12) Patent Application Publication
(10) Pub. No.: US 2015/0060728 A1
(43) Pub. Date: Mar. 5, 2015

(54) REAGENT FOR ENHANCING GENERATION OF CHEMICAL SPECIES

(71) Applicant: TOYO GOSEI CO., LTD., Inzai-shi (JP)

(72) Inventors: Satoshi Enomoto, Inzai-shi (JP); Takashi Miyazawa, Inzai-shi (JP)

(21) Appl. No.: 14/476,607

(22) Filed: Sep. 3, 2014

Related U.S. Application Data
(60) Provisional application No. 61/959,909, filed on Sep. 5, 2013.

(51) Int. Cl. C07C 49/84; C07C 43/295; C07C 43/263; C07D 309/12

(52) U.S. Cl. CPC C07C 49/84; C07D 309/12; C07C 43/295; C07C 43/263; H01L 2031/0344
USPC 252/183.11; 549/416; 568/648; 568/609; 568/333

(57) ABSTRACT

Described is a reagent that enhances acid generation of a photoacid generator and a composition containing such a reagent.

### CROSS-REFERENCE TO RELATED APPLICATION

This application claims the benefit under 35 U.S.C. §119(e) of U.S. Provisional Patent Application Ser. No. 61/959,909 filed on Sep. 5, 2013, the disclosure of which is hereby incorporated herein in its entirety by this reference.

### TECHNICAL FIELD

Several aspects of the disclosure relate generally to chemistry and more particularly to compositions that enhance generation of a chemical species such as an acid and/or base even if an inefficient phenomenon is utilized for the generation. Described herein is a reagent that enhances acid generation of a photoacid generator and a composition containing such a reagent.

### BACKGROUND

There have been attempts to form three-dimensional objects by utilizing two photon absorptions of materials. A method for forming three-dimensional objects by utilizing two photon absorptions of materials is disclosed in U.S. Pat. No. 5,914,807 to Downing (Jun. 22, 1999), the contents of the entirety of which is hereby incorporated herein by this reference.

### SUMMARY OF THE DISCLOSURE

Described is a first compound capable of generating a first chemical species, wherein the first chemical species is capable of emitting an electron or receiving an electron.

With regard to the first compound, it is preferred that the first compound has at least one aryl group because such aryl group can form an orbital able to interact with an orbital of another compound or an intermediate. Such orbital is involved with transfer of energy or electron.

With regard to the first compound, it is preferred that the first chemical species is capable of emitting an electron by an excitation of the first chemical species.

With regard to the first compound, it is preferred that the first chemical species be formed by having its hydrogen atom abstracted by a first reactive intermediate.

Also described is a second compound that is a derivative of the first compound. Preferably the second compound has at least one aryl group since such an aryl group can form an orbital able to interact with an orbital of another compound or an intermediate. Such an orbital is involved with transfer of energy or electrons. With regard to the second compound, it is preferred that the protecting group is a protecting group for a hydroxyl group.

Further described is a third compound capable of emitting an electron or accepting an electron by excitation of the third compound.

Still further described is a fourth compound that is a derivative of the third compound.

With regard to the third compound, it is preferred that the lowest singlet excited state of the third compound has pi-pi* nature.

Described is a composition that comprises the first compound and the third compound.

Preferably the composition further includes a fifth compound capable of generating an acid triggered by a light irradiation. Preferably, the fifth compound is capable of generating the acid by receiving an electron from the first chemical species or the third compound.

Preferably the composition further comprises a sixth compound that is to be excited by non-resonant two-photon (NRTP) excitation with a light, which cannot excite the sixth compound by one-photon transition.

Preferably, the composition further includes a sixth compound, which is to form a second reactive intermediate by having the composition excited by an electromagnetic ray or a particle ray.

With regard to the composition, it is preferred that the second reactive intermediate is capable of reacting with the first compound.

With regard to the composition, it is preferred that the second reactive intermediate be a radical.

A method for fabricating a device hereof includes placing any one of the compositions hereof and generating the second reactive intermediate.

Preferably, the method further includes performing an excitation of the third compound after the generating of the second reactive intermediate.

With regard to the method, preferably the generation of the second reactive intermediate is carried out by non-resonant, two photon (“NRTP”) excitation of the sixth compound.

A first compound hereof generates a first chemical species by reacting with a second chemical species, such as a radical and/or ion.

A precursor generates a third chemical species by reacting with the first chemical species, accepting an electron from the first chemical species, or donating an electron to the first chemical species.

A second compound hereof generates a third compound by reacting with the third chemical species, accepting an electron from the third or chemical species, or donating an electron to the third or chemical species.

The third compound can act as a catalyst or enhancer to stimulate a reaction on its own or receiving an energy such as electromagnetic ray, particle ray and heat. A latent image is formed through the process to the formation of the third compound. A composition relevant to an aspect of the disclosure contains the first compound and the second compound.

Since the second chemical species can be formed by providing such composition with energy such as electromagnetic ray, particle ray and heat, supply of energy to the composition can be utilized as a trigger for formation of the latent image.

Especially, the composition is very useful for a series of chemical processes even if an inefficient phenomenon, such as photoreaction induced by excitation by a non-resonant multi-photon process, or a light with low intensity is utilized for the series of chemical processes since the third compound formed in the composition can act as a catalyst or enhancer that enhances a desired reaction utilized for the chemical processes.

It is preferred that the first compound has a hydrogen atom that can be easily abstracted. Typically, the first compound has hydrogen on a sp3 carbon atom directly connected to an atom of an element other than carbon such as silicon, germanium, stannum, boron, phosphorus, and arsenicum, sp2 carbon atom, or sp carbon atom.

A typical example of the first compound is alcohol having at least one aryl group connected to a carbon atom bonded to the hydroxyl group and a hydrogen atom connected to the carbon atom or its derivative having a protecting group for the hydroxyl group. More typically, the first compound is monoaryl methanol or its derivative having a protecting group for the hydroxyl group. A compound containing a carbon atom, a silyl group connected to the carbon atom, a hydrogen atom connected to the carbon atom is an example of the first compound.

Other examples of the first compound are compounds having hydrogen on a sp3 carbon atom connected to an atom of element other than carbon such as silicon, germanium, stannum, boron, phosphorus, and arsenicum through one carbon atom.

Other examples of the first compound are compounds having hydrogen on an atom of element other than carbon such as silicon, germanium, stannum, boron, phosphorus, arsenicum, oxygen, and sulfur.

Typically, the first chemical species of which a typical example is ketyl radical is generated from the first compound by having its hydrogen atom abstracted by the second chemical species such as aryl radical or alkyl radical generated in the composition by supply of energy to the composition. It is preferred that the first chemical species has a reducing ability for reducing the precursor. The first chemical species may have an elimination group or atom that can be removed in conjunction with reduction of the precursor.

The precursor generates the third chemical species such as acid or base by receiving an electron from the first chemical species. A reaction of the third chemical species with the second compound results in the third compound. An example of such reaction is deprotection reaction by acid.

Typically, the second compound is a compound having a hetero atom such as oxygen, sulfur, and nitrogen and at least one aryl group connected to a carbon atom bonded to the hetero atom or its derivative having a protecting group for a substituent containing the hetero atom. More typically, the second compound is a derivative of diaryl ketone having a protecting group for the carbonyl group.

The third compound enhances a reaction such as generation of acid from the precursor by donating an electron to the precursor or receiving an electron from the precursor triggered by feeding of energy to the third compound or the composition. It is preferred that the third compound has a pi-conjugated system containing at least one aromatic ring or at least one multiple bond. Typically, the third compound may have at least two aromatic rings and at least one multiple bond that is conjugated with at least one of the at least two aromatic rings.

The third compound may have at least one electron-donating substituent such as alkoxy, alkylthio, arylthio, alkyl amino, and aryl amino on at least one aromatic ring. Examples of typical multiple bonds included in the third compound are carbon-oxygen double bonds, carbon-carbon double bonds, carbon-carbon triple bonds, carbon-nitrogen double bonds, carbon-nitrogen triple bonds, and carbon-sulfur double bonds. More typically, the third compound is a diaryl ketone having at least one electron-donating group on the aromatic ring.

It is preferred that the lowest excited state of the third compound have a singlet pi-pi* nature because such singlet excited state has a relatively longer lifetime to transfer its electron to the precursor and has relatively low reactivity. A compound having a triplet pi-pi* nature can be used as the third compound because the possibility of electron donation to the precursor or electron reception from the precursor can increase due to its longer lifetime compared to a singlet pi-pi* excited state. A compound having a triplet n-pi* nature can be used as the third compound because the third compound can react with precursor due to its high reactivity.

### BRIEF DESCRIPTION OF THE DRAWINGS

In the drawings, which illustrate what is currently considered to be the best mode for carrying out the invention:

FIG. 1 shows an irradiation system for NRTP excitation;

FIG. 2 shows an irradiation system for full radiation of the latent image; and

FIG. 3 shows a reaction scheme of a typical composition related to an aspect of the disclosure.

### DETAILED DESCRIPTION

Described is a compound that generates a chemical species, wherein the chemical species emits or receives an electron. The chemical species may be able to emit the electron via excitation of the chemical species. The chemical species may be formed by having its hydrogen atom abstracted by a first reactive intermediate. The compound may further have a protecting group (e.g., for a hydroxyl group).

Also described is a compound that, upon excitation, emits or accepts an electron. In such a compound, the lowest singlet excited state of the third compound preferably has a pi-pi* nature. This compound may further have a protecting group.

The invention is further described by the following illustrative examples.

Experimental Procedures:

### Synthesis of 2-[bis-(4-methoxy-phenyl)-methoxy]-tetrahydropyran (Compound 1)

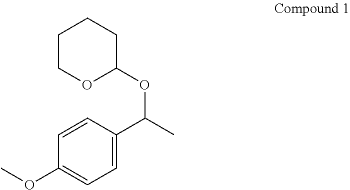 [SMILES: COc1ccc(C(C)OC2CCCCO2)cc1]

2.75 g of 2H-dihydropyran and 0.74 g of pyridinium p-toluenesulfonate are dissolved in 30.0 g of methylene chloride. 2.0 g of bis-(4-methoxy-phenyl)methanol (Compound 2) dissolved by 8.0 g of methylene chloride is added dropwise to the mixture containing 2H-dihydropyran and pyridinium p-toluenesulfonate over 30 minutes after that the mixture is stirred at 25 degrees Celsius for 3 hours. Since then, the mixture is further stirred after addition of 3% aqueous solution of sodium carbonate and then extracted with 20.0 g ethyl acetate. The organic phase is washed with water. Thereafter, ethyl acetate is distilled away. Thereby, 1.99 g of 2-[bis-(4-methoxy-phenyl)-methoxy]-tetrahydropyran is obtained.

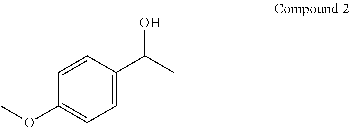

### Synthesis of bis-(4-methoxy-phenyl)-dimethoxymethane (Compound 3)

2.0 g of 4,4′-dimethoxy-benzophenone, 0.05 g of bismuth (III) trifluoromethanesulfonate and 5.7 g of trimethyl orthofomate are dissolved in 5.0 g of methanol. The mixture is stirred at reflux temperature for 42 hours. Since then, the mixture is cooled to 25 degrees Celsius and further stirred after addition of 5% aqueous NaHCO₃ solution. Then extracted with 30 g ethyl acetate and the organic phase is washed with water. Thereafter, ethyl acetate is distilled away, and the resultants are purified by silica gel column chromatography (ethyl acetate:hexane=1:9). Thereby, 1.71 g of bis-(4-methoxy-phenyl)-dimethoxymethane is obtained.

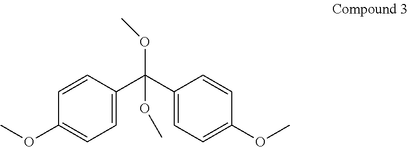

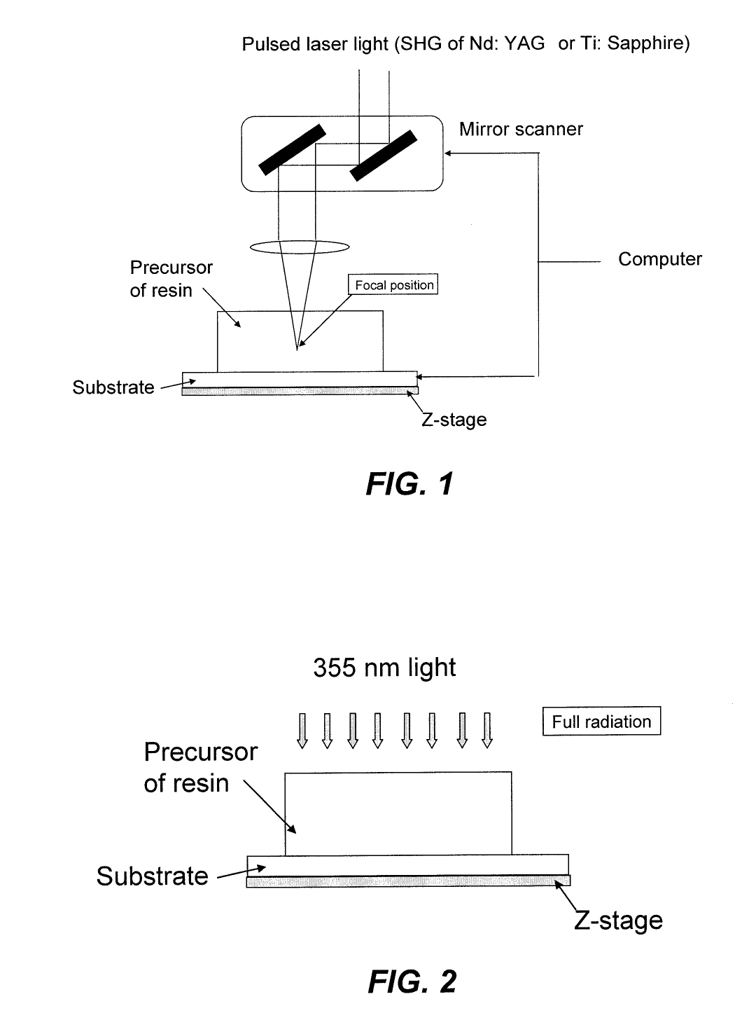

A composition is prepared by dissolving Compound 1, Compound 3, PAG-A and 4-bromostilbene in cyclohexene oxide. The composition is used as a precursor of resin. The composition is put on a substrate on which is placed on Z-stage to faun a coating film. A latent image is formed by irradiating the coating film using the irradiation system shown in FIG. 1. A 532 nm picosecond laser pulse, which is the second harmonic generation (SHG) of an Nd:YAG laser, is the light for formation of the latent image. The latent image is formed three-dimensionally by controlling focal positions by mirror scanner and Z-stage on which the precursor of resin is placed.

After the latent image is formed, full radiation using a 355 nm UV light, which is the third harmonic of the Nd:YAG laser, is carried out as shown in FIG. 2. A three-dimensional object or device is obtained by rinsing unreacted precursor off.

Since the precursor does not absorb the 532 nm pulsed light by direct one-photon transition, the precursor at a desired depth can be irradiated with the 532 nm pulsed light. Since the latent image formation uses non-resonant two photon (NRTP) excitations with a light that cannot excite the precursor by one-photon transition, the efficiency of reaction increases with the square of the intensity of the 532 nm pulsed light. Therefore, a high contrast is obtained.

In contrast, NRTP is not efficient. To overcome the inefficiency of NRTP exaction, full radiation with 355 nm light is used.

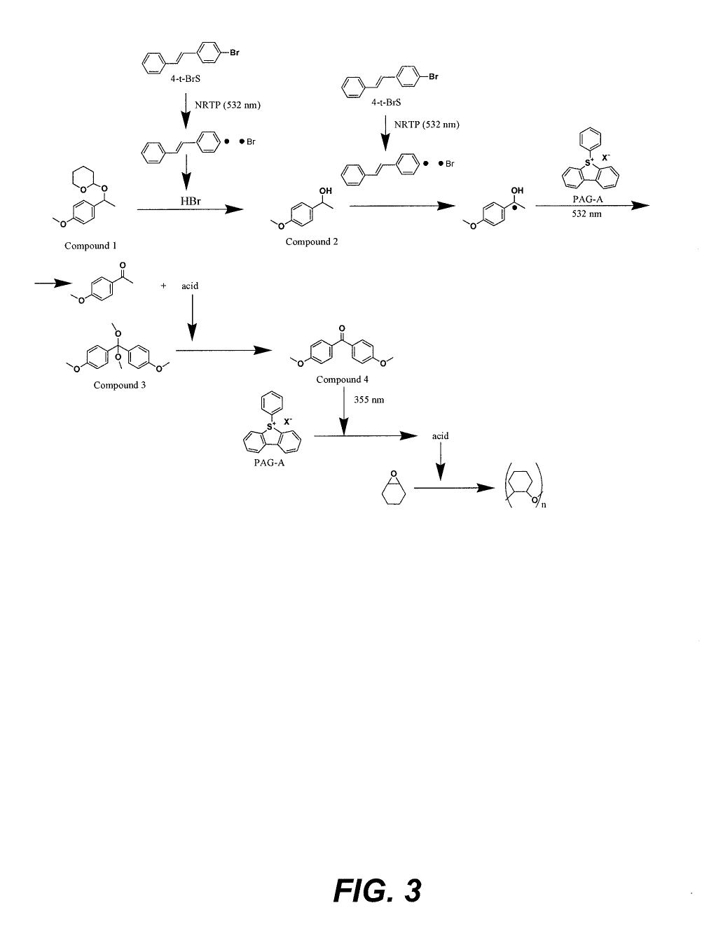

FIG. 3 shows an entire reaction scheme of the precursor. The NRTP excitation of 4-bromostilbene results in hydrogen bromide (HBr), which reacts with Compound 1 to form Compound 2 by deprotection reaction. Compound 2 gives a corresponding ketyl radical by having a hydrogen atom on the carbon atom connected to the hydroxyl group by the radical generated by the NRTP excitation of 4-bromostilbene.

The ketyl radical donates an electron to PAG-A by excitation of the ketyl radical by the 532 nm light. PAG-A generates acid by receiving the electron from the ketyl radical. Compound 3 reacts with the generated acid to form a corresponding ketone (Compound 4). In other words, Compound 4 is formed through the process of formation of the latent image using the NRTP excitation.

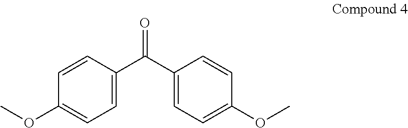

After the NRTP excitation is carried out, the full radiation with the 355 nm light is carried out to excite Compound 4. The excited Compound 4 donates an electron to PAG-A. PAG-A generates acid by accepting the electron from the excited Compound 4.

Instead of Compound 1, Compound 2 is also used as a starting material. Compounds 5, 6, 7, and 8 and their derivative having a protecting group for the hydroxyl groups are also used instead of Compound 1 as preferable examples. Such examples easily form reactive intermediates having electron-donating nature such as ketyl radical.

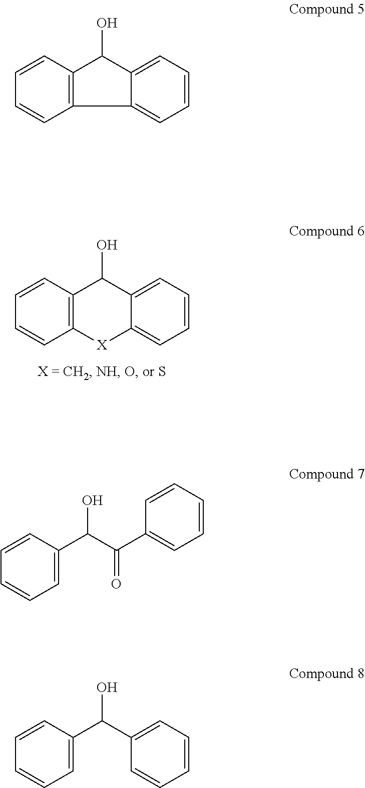

Instead of Compound 3, Compounds 9, 10, and 11 are also used as preferable examples:

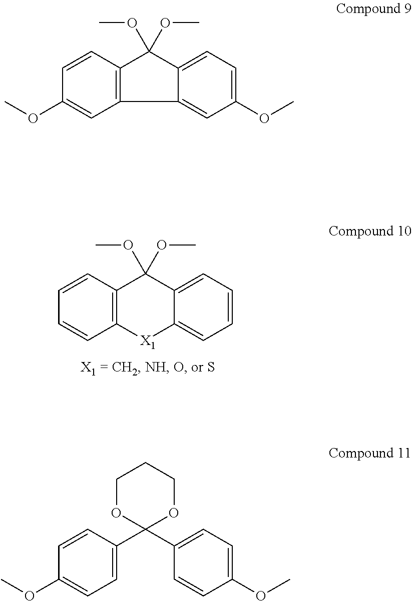

Such preferable examples have dissociable groups that are to be deprotected by acid. Their conjugated system is extended by the deprotection reactions. Compounds formed by the deprotection reactions from such preferable examples have electron-donating nature by absorbing a light.

What is claimed is:

1. A first compound able to generate a first chemical species, wherein the first chemical species is able to emit or receive an electron.

2. The first compound of claim 1, wherein the first chemical species is able to emit an electron via excitation of the first chemical species.

3. The first compound of claim 1, wherein the first chemical species is to be formed by having its hydrogen atom abstracted by a first reactive intermediate.

4. A compound that is a derivative of the first compound of claim 1, characterized by having a protecting group.

5. The compound of claim 4, wherein the protecting group is a protecting group for a hydroxyl group.

6. A third compound, which is able to emit an electron or accept an electron by having it excited.

7. The third compound of claim 6, wherein the lowest singlet excited state of the third compound has pi-pi* nature.

8. A fourth compound that is a derivative of the third compound of claim 6, characterized by having a protecting group.

9. A composition comprising:
a first compound able to generate a first chemical species, wherein the first chemical species is able to emit or receive an electron; and
another compound that is able to emit an electron or accept an electron by having it excited.

10. The composition of claim 9, further comprising:
a fifth compound, which generates an acid triggered by light irradiation.

11. The composition of claim 10, wherein the fifth compound is able to generate acid by receiving an electron from the first chemical species or the another compound.

12. The composition of claim 10, further comprising:
a sixth compound, which is to be excited by non-resonant two-photon excitation.

13. The composition of claim 10, further comprising:
a sixth compound, which is able to form a second reactive intermediate by having the composition excited by an electromagnetic ray or a particle ray.

14. The composition of claim 13, wherein the second reactive intermediate is able to react with the first compound.

15. The composition of claim 13, wherein the second reactive intermediate is a radical.

16. A method for fabricating a device, the method comprising:
placing the composition of claim 13; and
generating the second reactive intermediate,
so as to fabricate a device.

17. The method according to claim 16, further comprising:
performing an excitation of the another compound after generating of a second reactive intermediate.

18. The method according to claim 16, wherein generating the second reactive intermediate is carried out by a non-resonant two-photon excitation of a sixth compound, which sixth compound is to be excited by non-resonant two-photon excitation or which sixth compound is able to form the second reactive intermediate by having the composition excited by an electromagnetic ray or a particle ray.

19. The first compound of claim 1, having at least one aryl group.

20. The compound of claim 4, having at least one aryl group.
